(19) United States
(12) Patent Application Publication
(10) Pub. No.: US 2023/0001086 A1
(43) Pub. Date: Jan. 5, 2023

(54) MEDICAL FLUID PUMP COMPRISING DISPLAY UNIT

(71) Applicant: B. Braun Melsungen AG, Melsungen (DE)

(72) Inventors: Jan Schwarz, Melsungen (DE); Jana Roeper, Kassel (DE); Maria Buerger, Malsfeld (DE); Christoph Erlen, Kassel (DE); James Richard Green, Chester (GB); Mike Hoellerich, Koeln (DE)

(21) Appl. No.: 17/856,092

(22) Filed: Jul. 1, 2022

(30) Foreign Application Priority Data
Jul. 1, 2021 (DE) 20 2021 103 554.4

(51) Int. Cl. A61M 5/172; G06F 3/14; G06F 3/02

(52) U.S. Cl. CPC A61M 5/172; G06F 3/1423; G06F 3/02; A61M 2205/502; A61M 2205/505; A61M 2205/18; A61M 2205/3386

(57) ABSTRACT

A medical fluid pump of the infusion or syringe pump design includes a fluid pump housing which includes a display unit, in particular a display or touch display, with a plurality of display panels that are prepared and configured to display at least one respective function parameter of the fluid pump, including at least one setting parameter which can be set directly by a user of the fluid pump and a plurality of operating parameters which cannot be set directly by the user. The representation of the at least one setting parameter stands out visually against the operating parameters, such as by displaying the at least one setting parameter in an enlarged size compared to the operating parameters.

### CROSS-REFERENCE TO RELATED APPLICATION

This application claims priority under 35 U.S.C. § 119 to German Application No. 20 2021 103 554.4, filed Jul. 1, 2021, the content of which is incorporated by reference herein in its entirety.

### FIELD

The disclosure relates to a medical fluid pump, preferably an infusion pump or a syringe pump, for delivering a fluid/a liquid including a drug/a fluid drug/an infusion solution comprising a display unit.

### BACKGROUND

Known fluid pumps include display units or displays or screens which serve as a graphical user interface (GUI). Via the display unit, a user can enter instructions into the fluid pump and information about the fluid pump can be displayed to the user. For example, via the display unit itself or via function keys coupled thereto or the like, the fluid flow rate and a dosing rate of the delivered fluid can be set. Information which can be represented via the display unit include, inter alia, fluid pump parameters such as the delivery rate and, resp., the dosing rate of the fluid, the fluid volume delivered by the fluid pump, or the like.

Basically, medical devices comprising a display unit are known from the state of the art. For example, EP 2 973 101 B1 discloses an extracorporeal blood treatment system comprising plural pumps and comprising a display having a graphical user interface via which, inter alia, delivery and, resp., dosing rates of plural fluid reservoirs of the individual pumps as well as operating surfaces for users via which he/she can perform particular settings on the extracorporeal blood treatment system are displayed. Thus, a plurality of information is displayed to the user via the display known from EP 2 973 101 B1.

In addition, from CN 201431672 Y a medical fluid pump comprising a display is known. Via function keys formed separately from the display and a keyboard, a user can set and, resp., adjust setting parameters of the fluid pump which are shown via the display. Moreover, the display can display further information such as information about a patient who receives a fluid from the fluid pump.

In this way, also the display according to CN 201431672 Y displays a plurality of information.

Hence, it is difficult for a user when looking at the display to differentiate, within short time, out of the plurality of information the parameters important to him/her from the remaining information. The parameters important to users usually are those parameters which the users themselves can directly set, namely the so-called setting parameters.

### SUMMARY

Therefore, it is an object of the present disclosure to reduce or eliminate the problems of the state of the art. In particular, the present disclosure is intended to provide a medical fluid pump comprising a display unit which enables a user of the fluid pump to quickly and easily differentiate the setting parameters shown by the display unit from the other shown information.

The disclosure relates to a medical fluid pump of the infusion or syringe pump design comprising a fluid pump housing which includes a display unit, in particular a display or touch display. The display unit shows, in particular when fluid is delivered, a plurality of display panels which are prepared and configured to display at least one respective function parameter of the fluid pump. The function parameters include at least one setting parameter which can be set directly by a user of the fluid pump and a plurality of operating parameters which cannot be set directly by the user. The representation of the at least one setting parameter stands out visually compared to the operating parameters.

In other words, the disclosure relates to a medical fluid pump of the infusion or syringe pump design comprising a fluid pump housing which includes a display preferably on its one end face. The display shows a plurality of display panels each displaying at least one function parameter of the fluid pump. A function parameter may be a setting parameter which can be set directly by a user of the fluid pump or may be an operating parameter which cannot be set directly by the user. The representation of the setting parameter itself and, resp., the representation of the display panel with the setting parameter is visually outstanding or prominent compared to the representation of the operating parameters and, resp., the representation of the display panels with the operating parameters. The display panels are particularly displayed while fluid is delivered.

This offers the advantage that users can directly and immediately view, with a quick glance at the display unit, the setting parameter(s) important to them.

In a preferred embodiment, the at least one setting parameter is displayed enlarged vis-à-vis the remaining function parameters. Alternatively, the at least one setting parameter could also be displayed in a different clearly perceptible color or bolder than the other function parameters. It would also be possible for the at least one setting parameter to be flashing in contrast to the other function parameters. As an alternative, it would also be possible for the display panel displaying the at least one setting parameter to show a clearly perceptible color different from the remaining display unit. Advantageously, the color of the setting parameter should stand out clearly against the color of the corresponding display panel.

The afore-shown options for optically emphasizing the setting parameters compared to the operating parameters can be realized particularly easily and are effective.

A preferred embodiment provides that the fluid pump has, in the vicinity of the display unit, in particular directly laterally from the display unit, a number of function keys, in particular rotary knobs or push buttons, via which the fluid pump can be controlled/operated. The display panel which displays the at least one setting parameter is at a maximum distance from the function keys, i.e., on the side of the display panel which is opposite to the side of the display panel where the function keys are placed.

This arrangement of the function keys relative to the positioning of the at least one setting parameter and, resp., the display panel thereof offers the advantage that users can view the setting parameter, also when the function keys are operated, not just concealed by their hand, but can continuously view the setting parameter.

Further preferably, the display unit can include, in particular when fluid is delivered, apart from the number of display panels additionally or alternatively to the function keys, a number of control panels via which users of the fluid pump themselves can make changes on the display unit and/or can control function parameters and/or further parameters of the fluid pump which are different from the function parameters displayed in the display panels.

When the fluid pump includes, apart from the function keys, control panels on the display unit, particular functions for operating the fluid pump can be distributed to the function keys and the control panels. It would also be conceivable that, if at least one of the function keys does no longer work (properly), at least one of the control panels takes over the function thereof. On the other hand, it would be conceivable that the function keys take over specific functions of the control panels if the display unit fails.

Via the control panels, for example a view can be changed while fluid is delivered so that one or more of the display panels display a different parameter. The function keys can include, for example, a stop function for stopping the fluid delivery, an on-/off function for turning the fluid pump on or off, or a menu function for changing the representation of a view during fluid delivery with display panels and/or control panels to a general home view. In the home view, various buttons are available on the display which permit, inter alia, to adjust the parameters of delivery rates or to set up a new therapy or to change pressure parameters and/or to change alarm volumes or display brightness.

It is desirable that the control panels within the display unit are at a maximum distance from the display panel with the at least one setting parameter.

This arrangement of the control panels relative to the positioning of the setting parameter offers the advantage that a user does not cover the setting parameter with his/her hand, even while operating the control panels, but can view the setting parameter continuously.

In one embodiment, the setting parameter is a dosing rate or a fluid delivery rate of a fluid delivered by the fluid pump. Accordingly, it is sufficient to set one of said parameters, as the other is directly related thereto.

These parameters are particularly relevant to operating a medical fluid pump. Hence, it is particularly important for a user of the medical fluid pump to be able to quickly and reliably view the dosing rate and, resp., the fluid delivery rate of a fluid delivered by the fluid pump.

The operating parameters can be a fluid volume delivered by the fluid pump, a residual fluid volume remaining in the fluid pump, a fluid concentration of the fluid delivered by the fluid pump and, in the event of inoperability of the fluid pump, for example if there is no more fluid in the fluid pump, an alarm or alert.

Further operating parameters can be a remaining time of the fluid pump depending on the residual fluid volume remaining in the fluid pump, a fluid volume used during a current pump cycle, the name of the fluid or drug used, the concentration thereof, preset dosing limits of the fluid or drug, the status of the fluid delivery (active or stopped), information about a status of a communication unit (active or inactive, such as WIFI is on/off) that is connected to the fluid pump, or else a battery life of a battery of the fluid pump or the charge level thereof.

When this plurality of operating parameters is shown on the display unit, it is particularly helpful for the user when the relevant setting parameters stand out visually against the other function parameters.

There is the further option of the function parameters being displayed in the form of numerical values and/or being graphically displayed.

In the case that one or more function parameters are displayed as a decimal number (containing a comma), the integer place/decimal point is preferred to be displayed larger than the decimal places.

This improves the legibility of the correspondingly shown function parameters for the user.

Advantageously, the fluid pump further includes sensors and a control unit to determine the function parameters out of the parameters detected by the sensors.

### BRIEF DESCRIPTION OF THE DRAWING FIGURES

FIG. 1 is a perspective view of a medical fluid pump in the form of a syringe pump comprising a display unit;

FIG. 2 is a first example of a view of the display unit;

FIG. 3 is a second example of a view of the display unit;

FIG. 4 is a third example of a view of the display unit;

FIG. 5 is a fourth example of a view of the display unit;

FIG. 6 is a fifth example of a view of the display unit; and

FIG. 7 is a sixth example of a view of the display unit.

### DETAILED DESCRIPTION

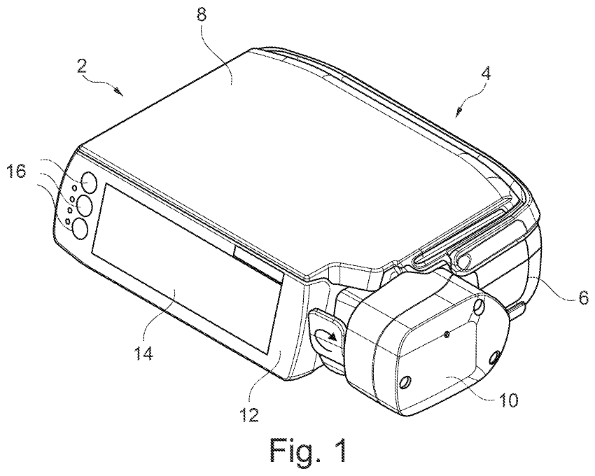

FIG. 1 illustrates a medical fluid pump according to the invention configured as a syringe pump 2 in this case. The basic functioning of the syringe pump 2 is sufficiently described in EP 0 566 825 A1 so that here a detailed description of the syringe pump 2 is omitted.

The syringe pump 2 shown in FIG. 1 includes a pump housing 4 having a housing lower shell 6 and a housing upper shell 8. A drive head 10 is connected to the syringe pump 2 to be movable relative to the housing 4. On the front face of the syringe pump 2, a front flap 12 is provided and configured. The front flap 12 is hinged to the housing lower shell 6 to be foldable via hinges. The front flap 12 further includes a display unit or display 14. In addition, the front flap 12 includes three function keys 16 on the left side next to the display unit 14. The possible functions of the function keys were explained in the foregoing.

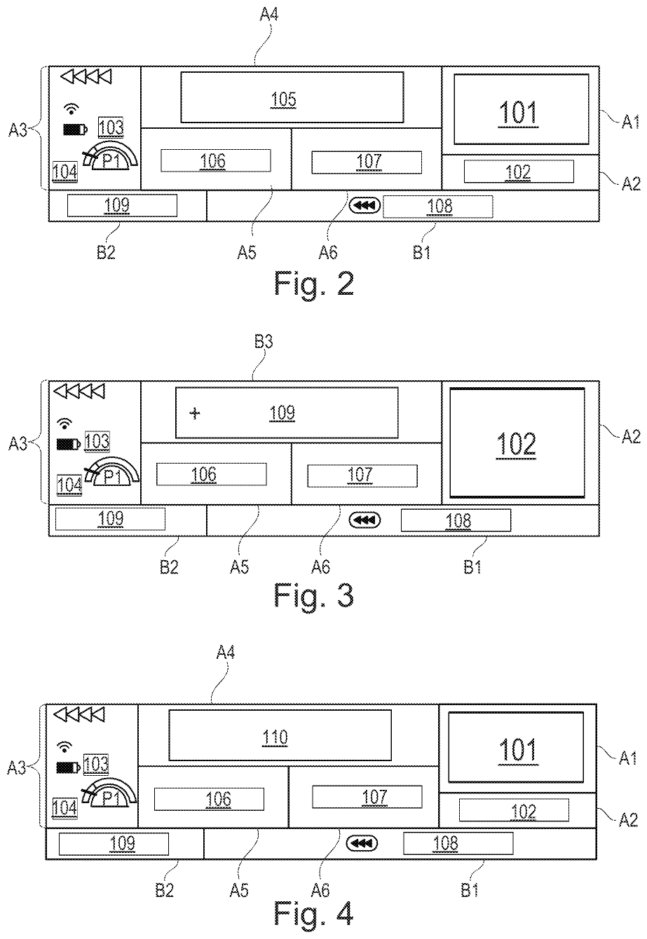

The following FIGS. 2 to 7 are views which appear only while the fluid pump is actually delivering fluid or an infusion is carried out (fluid delivery view). Usually, the display panels show a text describing the function parameter. The function parameter itself is represented in the respective panel as a numerical value (with a unit) and/or as a graph. However, display panels can additionally or alternatively also represent symbols. A user can basically have a different operating parameter displayed by touching, specifically by tapping or clicking on, a display panel with an operating parameter in said display panel. By touching, specifically by tapping or clicking on, a display panel with a setting parameter, a user can change the setting parameter.

All control panels can be operated by a user manually, in particular by touching/pressing the respective control panel. The respective functions of the control panel are represented by symbols and/or text. Plural display and/or control panels can be combined with each other at will to form a view, wherein it is desired that at least one setting parameter, in particular the dosing rate or the fluid delivery rate, is displayed in any case. The display panels and control panels are arranged in a tile-like manner in all Figures.

In the following description of the FIGS. 2 to 7 showing views of the display unit different from each other, basically the display panels are marked A and the control panels are marked B. All empty boxes shown in the FIGS. 2 to 7 with their respective reference symbol in the display panels are representative of the setting and operating parameters which can be presented here. The setting and operating parameters are usually indicated with their designation and the corresponding numerical value with a unit. As an alternative, said parameters can also be displayed with their designation and a graphical progression of the respective parameter. If a setting parameter is shown enlarged, the respective reference symbol is shown in a larger font size than the other reference symbols. In the control panels, the empty boxes with their respective reference symbol replace a text informing the users about the type of operation they can perform via the respective panel. The examples shown in FIGS. 2 to 7 of the distribution of the panels within the display unit 14 are merely exemplary and serve for illustration purposes only.

FIG. 2 is a first example of a distribution of display panels and control panels of the display unit 14. In this first example, the display unit 14 shows two display panels A1 and A2 each including a setting parameter. For example, A1 could indicate the current dosing rate 101 of a fluid delivered by the fluid pump and A2 could indicate the current fluid delivery rate 102 of the fluid delivered by the fluid pump which is directly related with the dosing rate. For example, 101 stands for “dosing rate: 50 mg/h” and 102 stands for “620 ml/h” or for “fluid flow rate: 20 ml/h”.

Moreover, FIG. 2 illustrates four display panels A3 to A6 with operating parameters. A3 displays in the form of a symbol whether or not the fluid pump delivers fluid. In addition, A3 shows in the form of a symbol the receiving state of a communication unit which is communicated with the fluid pump. A3 further shows a battery charge level of a battery of the fluid pump by a symbol and the resulting battery life 103 as a numerical value (with a unit). For example, 103 stands for “3.5 h”. A3 further shows the pressure 104 of the fluid pump by a symbol and a numerical value (with a unit). For example, 104 stands for 84 mmHg. A4 indicates a fluid concentration or drug concentration 105 of a delivered drug. For example, 105 stands for “basic drug concentration: 4 mg/50 ml”. A5 indicates a remaining time 106 of the fluid pump indicative of how long the fluid pump can still deliver the fluid with the present fluid volume in response to the setting parameter shown in A1 and, resp., A2. For example, 106 stands for “remaining time: 2 h 29 min 17 sec”. A6 displays the remaining volume/remaining fluid volume 107 indicative of the fluid volume still present in the fluid pump and varying over time and in response to the setting parameter. For example, 107 stands for “remaining volume: 9.34 ml of 310 ml”.

Further, in FIG. 2 two control panels B1 and B2 are shown. In the panel B1, 108 is representative of the text “bolus”, for example. By operating B1, in particular as long as a user presses the panel B1, exclusively during the already running fluid delivery of the fluid pump 2 a bolus menu is caused to be opened in which a desired drug bolus can be determined. As an alternative, via operating B1, a signal can be transmitted to the fluid pump so that it delivers a predetermined bolus.

In the panel B2, 109 stands for the text “change view”, for example. Hence, the view can be changed by operating B2. Consequently, in one or more display panels, other function parameters than before can be displayed and/or new control panels can be displayed. By operating B2, it can be changed between the views shown in the FIGS. 2 to 6, for example.

In FIG. 2, a total of two setting parameters (one in A1 and one in A2) are shown which are correlated. In this case, the numerical value of the setting parameter (or the reference symbol 101) in the panel A1 is displayed enlarged compared to all other information shown in FIG. 2 and, thus, is displayed with a larger font size. Consequently, the value in the panel A1 immediately catches the eye of a viewer or, resp., a user of the fluid pump and, thus, can be quickly differentiated visually from the remaining information.

The second example of a view of the display unit shown in FIG. 3 corresponds to the first example apart from the differences listed as follows. The panel A4 was replaced with a control panel B3 via which the user can select a further or other fluid or drug than before. For example, the reference symbol could stand for the text “add here a drug from the drug library”. Moreover, the panel A1 is missing in FIG. 3 as compared to FIG. 2. A2 now is the only display panel with a setting parameter and indicates the fluid flow rate. In A2, the numerical value of the fluid flow rate 102 (viz. the reference symbol 102 itself) is enlarged compared to the other information shown in FIG. 3, i.e., the numerical value is shown in a larger font size.

The third example of a view of the display unit shown in FIG. 4 basically corresponds to the first example. However, in the panel A4 next to the drug concentration also the name of the delivered drug is reproduced, for which the reference symbol 110 stands. For example, 110 stands for “DimenhyDrinate: 20 mg/250 ml”.

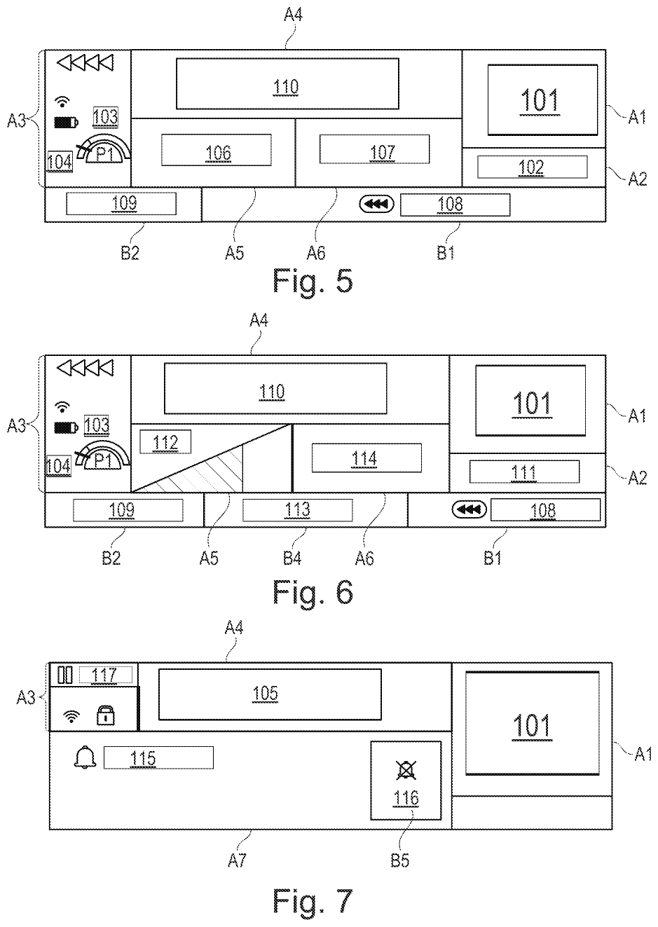

The fourth example of a view of the display unit shown in FIG. 5 corresponds to the third example according to FIG. 4 apart from the fact that in the panel A2 the maximum fluid delivery rate 102 is displayed.

The fifth example of a view of the display unit shown in FIG. 6 differs from the first example as follows. In FIG. 6, the panel A2 no longer indicates the fluid delivery rate but indicates the desired dosing rate 111. The panel A5 shows a graph of a function parameter. For example, a ramp-shaped start of a fluid delivery is graphically illustrated. The reference symbol 112 describes what is graphically illustrated (such as “ramp”). Further, FIG. 6 includes another control panel B4 via which the current ramp can be cancelled/stopped. The reference symbol 113 could stand for the text “stop ramp” here. A6 shows as a numerical value of how long said ramp will still be run. The reference symbol 114 stands for that.

FIG. 7 shows a sixth example of a view of the display unit in which an alarm or alert is shown in a display panel A7. For example, the reference numeral 115 stands for the text “fluid is empty”. The panel A7 with the alarm/alert stands out visually, such as in color, against the other panels. In the case of alarm, additionally an acoustic alarm can be triggered which can be stopped via another control panel B5. For example, the reference numeral 116 here stands for the text “mute”. As an alternative, the alarm notice can be minimized via the control panel B5. Said alarm/alert can inform, for example, about the fact that the fluid or drug is exhausted/empty, or that gas bubbles were detected in the fluid. If an alarm/alert is displayed, the fluid delivery of the fluid pump is stopped, which is indicated in the panel A3. Accordingly, the reference symbol 117 could stand for the text “stopped”. However, the fluid delivery can be resumed only when a user has eliminated the reason for the alarm.

In addition, the panel A3 shows the receiving state of the communication unit. Moreover, in A3 a symbol is displayed showing that the operation of the fluid pump is blocked at least via the display unit. The dosing rate is displayed in the panel A1, with the numerical value of the dosing rate being shown enlarged as compared to all remaining information. The current drug concentration is displayed in the panel A4.

The view shown in FIG. 7 cannot be selected independently by a user, but appears only if there is a reason for an alarm/alert and the fluid pump detects this with the aid of one or more sensors. It is basically also possible that only a pre-alarm is displayed, where the fluid delivery is not stopped.

1. A medical fluid pump having an infusion or syringe pump design, the medical fluid pump comprising a fluid pump housing having a display unit with a plurality of display panels configured to display at least one function parameter of the medical fluid pump, the at least one function parameter including at least one setting parameter that can be set directly by a user of the medical fluid pump and a plurality of operating parameters that cannot be set directly by the user, the plurality of display panels comprising a first display panel configured to display the at least one setting parameter in a manner that stands out visually from the plurality of operating parameters.

2. The medical fluid pump according to claim 1, wherein the medical fluid pump includes, in a vicinity of the display unit, a plurality of function keys via which the medical fluid pump is operable, the first display panel being at a maximum distance from the plurality of function keys.

3. The medical fluid pump according to claim 2, wherein the plurality of function keys are provided directly laterally from the display unit.

4. The medical fluid pump according to claim 2, wherein the plurality of function keys are configured as rotary knobs or push buttons.

5. The medical fluid pump according to claim 2, wherein the display unit comprises a plurality of control panels enabling the user to make changes on the display unit and/or to control function parameters and/or further parameters of the medical fluid pump that are different from the function parameters.

6. The medical fluid pump according to claim 5, wherein the plurality of control panels are located within the display unit at a maximum distance from the first display panel.

7. The medical fluid pump according to claim 5, wherein particular functions for operating the medical fluid pump can be distributed to the plurality of function keys and the plurality of control panels.

8. The medical fluid pump according to claim 5,
wherein at least one of the plurality of control panels is configured to take over a function of one of the plurality of function keys when said one of the plurality of function keys becomes inoperable, or
wherein the plurality of function keys are configured to take over functions of the plurality of control panels if the display unit becomes inoperable.

9. The medical fluid pump according to claim 1, wherein the at least one setting parameter is a dosing rate or a fluid delivery rate of a fluid delivered by the medical fluid pump.

10. The medical fluid pump according to claim 1, wherein the plurality of operating parameters are a fluid volume delivered by the medical fluid pump, a residual fluid volume remaining in the medical fluid pump, a fluid concentration of a fluid delivered by the medical fluid pump and an alarm or alert.

11. The medical fluid pump according to claim 1, wherein the at least one function parameter is displayed as a numerical value and/or are displayed graphically.

12. The medical fluid pump according to claim 1, wherein the at least one function parameter is displayed as a decimal number having an integer place and a decimal place, with the integer place displayed larger than the decimal place.

13. The medical fluid pump according to claim 1, wherein the medical fluid pump further comprises sensors and a control unit to determine the plurality of operating parameters from the sensors.

14. The medical fluid pump according to claim 1, wherein the display unit is a display or a touch display.

15. The medical fluid pump according to claim 1, wherein the at least one setting parameter is displayed enlarged compared to each of the plurality of operating parameters.

16. The medical fluid pump according to claim 1, wherein the at least one setting parameter is displayed in a different color than the plurality of operating parameters or displayed bolder than the plurality of operating parameters.

17. The medical fluid pump according to claim 1, wherein the at least one setting parameter is flashing while the plurality of operating parameters are not flashing.

18. The medical fluid pump according to claim 1, wherein the display panel displays a first color different from a second color displayed by the display unit.

19. The medical fluid pump according to claim 1, wherein the plurality of display panels comprise a plurality of second display panels, with the plurality of operating parameters displayed on the plurality of second display panels.

20. The medical fluid pump according to claim 19, wherein the first display panel and the plurality of second display panels are visually separated from each other by lines.

21. The medical fluid pump according to claim 19, wherein the first display panel displays exactly one of the at least one setting parameter, and the plurality of second display panels each display exactly one of the plurality of operating parameters.
